(19) United States
(12) Patent Application Publication
(10) Pub. No.: US 2017/0131288 A1
(43) Pub. Date: May 11, 2017

(54) Test Strip for Holding Reagents to Determine Blood Glucose Level

(71) Applicants: Indian Council of Medical Research, New Delhi (IN); Birla Institute of Technology and Sciences, Andhra Pradesh (IN)

(72) Inventor: Suman KAPUR, Andhra Pradesh (IN)

(21) Appl. No.: 15/318,772

(22) PCT Filed: Jun. 18, 2015
(86) PCT No.: PCT/IN2015/000251

§ 371 Date: Dec. 14, 2016

(30) Foreign Application Priority Data
Jun. 18, 2014 (IN) 1646/DEL/2014

(51) Int. Cl. G01N 33/66; G01N 33/52

(52) U.S. Cl. CPC G01N 33/66; G01N 33/521; G01N 2333/904

(57) ABSTRACT

This invention relates to a test strip for holding reagents to determine blood glucose level, said strip comprising a) a plasma separator membrane which is a composite membrane for separation of plasma from whole blood, said plasma separator membrane containing an enzyme mix specific to the analyte, said analyte being glucose, a substrate which reacts specifically with one of the products of the enzymatic reaction of the analyte with the enzyme mix, b) a reaction membrane containing an enhancer mix of metallic ions, which enhances the color developed due to the enzymatic reaction.

### FIELD OF THE INVENTION

The present invention relates to a test strip for determination of blood glucose level. This invention further relates to an assembly of reagents and glucose specific enzymes to test the blood glucose level in plasma. In particular, the described invention is a sequential arrangement of plasma separator and reagents, which produces a colored product, whose intensity is a function of the blood glucose concentration.

### BACKGROUND OF THE INVENTION

Glucose is the chief metabolic fuel for generation of energy in the body. The blood glucose level is chiefly controlled by insulin, a hormone secreted by β-cells of the islets of Langerhans located in the pancreas. The normal blood glucose level is in a range of 80-126 mg/dl. Rapid and accurate determination of blood glucose level is crucial for those suffering from diabetes. The higher plasma glucose level in diabetics give rise to severe health complications including diabetic retinopathy, diabetic foot, coma and in extreme cases even death. Self and rapid monitoring of blood glucose level by diabetics thus become crucial in determining drug dose, severity of the disorder and susceptibility of the patient to various other associated disorders.

At present, “test strips” are used frequently by diabetics for self determination of blood glucose level. A “test strip” carries all the essential reagents required to produce a measurable signal by reacting with the analyte of interest. Upon reaction with the analyte, in this instance glucose, a measurable signal is produced by reaction of glucose and reagents and produced signal is measured either amperometrically, or colorimetrically, with the current/color intensity is in proportion to the glucose level present in the plasma.

The reading device is usually a photometer, which measures the quantity of light reflecting from the particular body. In case of glucose measurement, the reflected light from a particular chormophore is translated into a glucose concentration using a predefined algorithm.

At the time of using a “test strip”, a drop of blood is placed on a defined area, which contains reagents and enzymes required to complete the assay. The reaction, which usually completes in 20-30 seconds, develops a colour which is measured using the provided glucometer.

Despite the fact that “test strip” model of glucose detection is widely used and universally accepted by diabetic for self and rapid determination of blood glucose, the present day system of self-monitoring blood glucose falls short of accuracy. The self-monitoring system has not been successful in producing consistent results for several reasons, including batch to batch variations in the thickness of reagent composition, storage temperature, improper plasma separation, insufficient availability of oxygen in the reaction area, degradation of enzymes/reaction components due to temperature variations and user to user variability.

U.S. Pat. No. 6,518,034 for a test strip for blood glucose evaluation discloses a test strip for use with a photometer for determining blood glucose concentration from a blood sample, said strip comprising: a substrate having a top surface and an opposing bottom surface, said substrate having an aperture formed therethrough, said aperture being adapted to receive and receiving a droplet of blood; a porous membrane having a top surface and an opposing bottom surface, said top surface of said membrane adhered to said bottom surface of said substrate so that said membrane is in registration with said aperture in said substrate, said porous membrane filters red blood cells from plasma in said blood; and a reagent carried on said bottom surface of said membrane, the interior of said membrane being substantially free of said reagent, said reagent reacts with glucose in blood plasma.

U.S. Pat. No. 4,281,062 to Kallis relates to a test for identification and determination of glucose. The patent discloses a test strip containing a metering zone, a reaction zone with glucose oxidase, and an indicator zone having at least one indicator signal zone of peroxidase and indicator applied in the form of stripes alternating with untreated intermediate spaces. The metering zone may be impregnated with a colorant, oxidizing, precipitating or buffering agent. The glucose oxidase is applied in an acid solution below the isoelectric point to prevent substrate inhibition by means of test fluid diffusion and may be in the form of an immobilized suspension. Together with a flow-inducing agent, the test strip allows a reaction not influenced by the time elapsed after sampling. Intermediate zones located between the reaction zone and the metering zone and/or the identification zone may be prepared with buffering substances. The test strip may be located within a capillary tube. However, the methods and the test strips are complicated and do not allow easy handling. Further, in view of the elaborate structures, the test strips are expected to be expensive particularly for the common man.

### OBJECTS OF THE INVENTION

It is therefore an object of this invention to propose a test strip for determination of blood glucose level, which is inexpensive.

It is a further object of this invention to propose a test strip for determination of blood glucose level, which gives consistent results.

Another object of this invention is to propose a test strip for determination of blood glucose level, which can be made from simple, readily-available materials.

These and other objects of the invention will be apparent to a reader on reading the ensuing description, in conjunction with the accompanying drawings.

### BRIEF DESCRIPTION OF THE ACCOMPANYING DRAWINGS

FIG. 1 is a front view of the test strip.

FIG. 2 is elevated view of the composite and circular disks upon which reagents and enzymes are immobilized.

FIG. 3 Graph showing the comparison between liquid assay and the Glucose estimating strips and nanoamperometric glucometer.

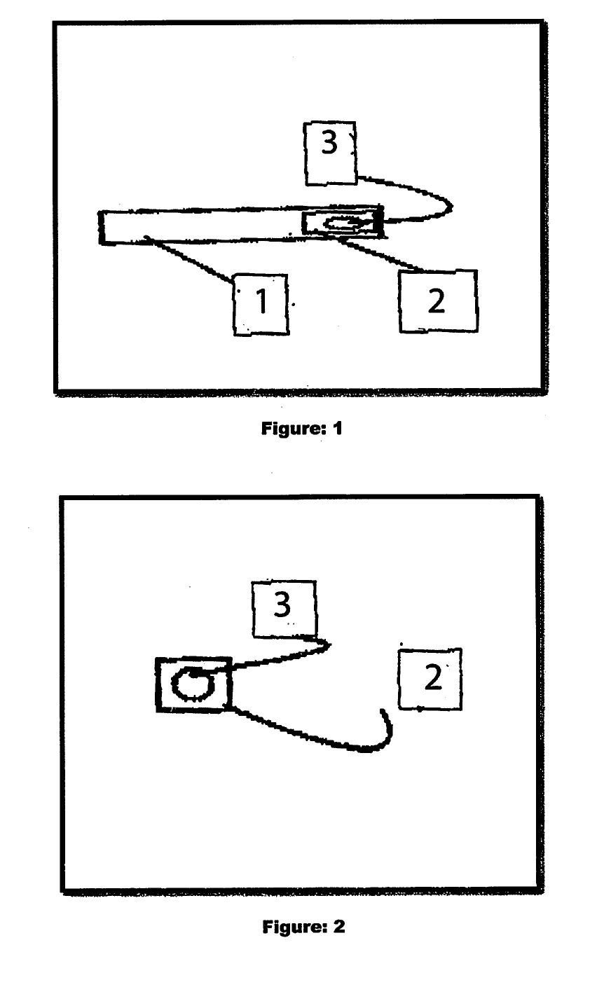

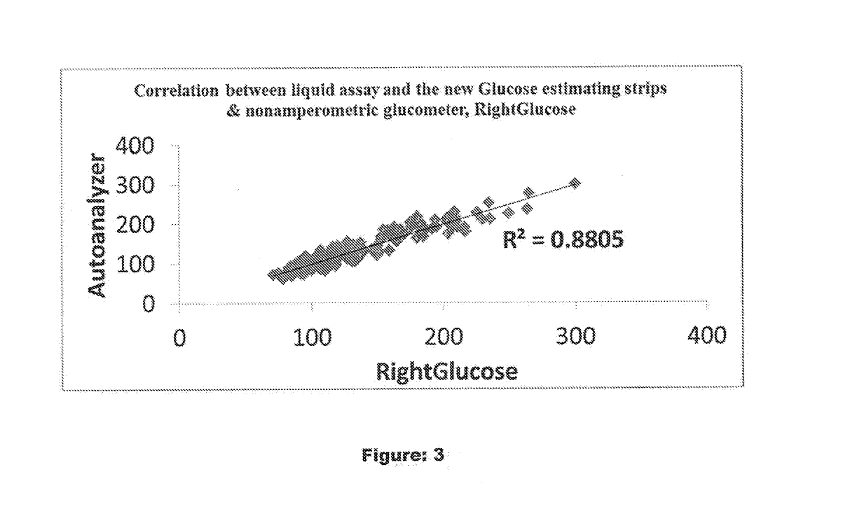

### DETAILED DESCRIPTION OF THE INVENTION

Thus according to this invention is provided a test strip for determination of blood glucose level in plasma.

In accordance with this invention, the test strip for blood glucose determination is made by assembling plasma separator, enhancer, enzyme mix and a chromogenic substrate.

The FIG. 1 represents the sequential arrangement of various components of the said test strip from the front.

The test strip comprises :

a) a plasma separator membrane which is a composite membrane for separation of plasma from whole blood, said plasma separator membrane containing

an enzyme mix specific to the analyte, said analyte being glucose,

a substrate which reacts specifically with one of the products of the enzymatic reaction of the analyte with the enzyme mix,

b) a reaction membrane containing an enhancer mix of metallic ions, which enhances the color developed due to the enzymatic reaction.

The composite membrane for separation of plasma i.e. the plasma separator is a membrane made of polysulphone, cellulose, nitrocellulose and glass fibres, preferably polysulphone. It has specific dimensions and it is and positioned in said test strip for the purpose of plasma separation from whole blood by entrapping cellular components specially the red blood cells. The polysulphone plasma separator prevents red blood cell leakage into the reaction area.

The enzyme mix is composed of glucose oxidase and peroxidase. The substrate is specific to one of the products of the enzymatic reaction, and develops a coloured product by reacting with one of the products of the enzymatic reaction. The substrate is selected from a group of 3,3′,5,5′-Tetramethylbenzidine, 2,2′-azino-bis(3-ethyl benzothiazoline-6-sulphonic acid), 3, 3′-diaminobenzidine and o-phenylenediamine.

The enhancer used in the test strip is used for enhancing the colour produced by the enzymatic reaction. The enhancer is present in dried form in the plasma separator. The enhancer is a metal salt selected from a group consisting of copper sulfate, nickel chloride, nickel ammonium sulfate and cobalt chloride and the ratio of metallic ions is specifically optimized for the substrate. The test strip consists of a porous fabric or material such as polyamide, polyolefin, polysulfone, or cellulose. The test strip is composed of two layers wherein plasma separator membrane is placed on top of the reaction membrane and is based on standard glucose oxidase-peroxidase enzymatic method wherein the final concentrations of both enzymes in the mixture are in a specified ratio based on the source of the enzyme.

The enzymes are mixed in a buffer of pH around 5 to 6 and a homogeneous mixture is prepared. In a preferred embodiment the peroxidase is horseradish peroxidase and the buffer is a citrate buffer.

After mixing thoroughly to obtain a homogeneous blend, the mix is spread on a cellulose sheet and dried at 20-25° C. The sheet is then cut into strips of the desired size and shape. The strips may be circular, square or rectangular. Next, the substrate is mixed with a buffer of pH between 5 to 6 such as citrate buffer and a homogeneous solution is made. This solution is spread on the cellulose sheet containing the enzyme mix, once the enzymes have dried completely. The cellulose strip containing the enzyme mix and the substrate is allowed to dry under dark conditions to avoid photooxidation of the substrate, to obtain the reaction membrane.

The enhancer mix is prepared by adding one or more salts in a citrate buffer of pH between 5 to 6 such as citrate buffer. The enhancer mix is spread on the plasma separator membrane and dried at a temperature of 20-25° C. To assemble the test strip, the reaction cellulose strip containing enzyme mix and substrate is placed below the plasma separator containing the enhancer mix to obtain a composite sandwich. The sandwich is again mounted on an additional membrane for additional structural integrity such as a HiPP strip. The membrane sandwich is protected from moisture and dust by covering with wide polyester mesh strips over them.

In the test strip (1), blood is placed on the membrane (2) which separates red blood cells from plasma thus allowing only clear plasma to react with enzymes immobilized on cellulose membrane (3). The reaction occurs in reaction membrane (3) that produces hydrogen peroxide, which reacts with its substrate present along with the said enzyme mix and produces a colored complex. Enhancer mix of heavy metals is present in dried form in membrane (2), which travels with plasma and intensifies the developed colour complex. The final reaction and intensification occur on membrane (2). The measurable signal, blue colour in this case, develops on the said reaction mixture. The chromogenic reaction produces color that is directly proportional to glucose concentration present in plasma. The enzyme mix comprises glucose oxidase and peroxidase which is immobilized on a cellulose membrane. Glucose present in the separated plasma reacts with enzyme mix to produce hydrogen peroxide which forms a light brown color complex with said chromogenic substrate. At this stage, enhancer mix present in plasma separation membrane turns developed brown color into intensified blue color complex. The final blue color is a function of glucose concentration present in plasma. The invention will now be explained in greater detail with the help of the following non-limiting examples.

### Example 1

The test strip is composed of two layers wherein plasma separation membrane is placed on top of a reaction membrane and is based on standard glucose oxidase-peroxidase enzymatic method wherein the final concentrations of both enzymes in the mixture are in a specified ratio based on the source of the enzyme. For example 625 units horseradish peroxidase and 2,500 units glucose oxidase in 10 mL of citrate buffer, pH 5.4 were mixed arid a homogeneous mixture of the enzymes was prepared. After mixing to ensure a homogeneous blend, the said enzyme mix was homogenously spread onto a cellulose sheet and dried at 22° C. The rows were then cut into strips of the desired size. Next, 25 mg of 3, 3′-diaminobenzidine was mixed with 10 ml of citrate buffer pH 5.4 and a homogenous solution of the chromogen is made. This chromogenic mix is spread on cellulose paper once the enzyme mix has been dried completely and allowed to dry under dark conditions. Then, enhancer mix was prepared by adding Cobalt Chloride (2%) and Nickel chloride (2%) in 4:1 ratio in citrate buffer, pH 5.4. The plasma separator membrane was quenched with the enhancer mix and dried at 22° C. To assemble the glucose test strip, cellulose disc (4 mm×4 mm) containing enzyme mix specific for analyte, in this case glucose, was placed below the said plasma separator containing enhancer mix (5 mm×5 mm). The sandwich of the discs is mounted on a HiPP strip of 5 mm×6 cm for added structural integrity. The membrane sandwich is protected from moisture and dust by a covering of 5 mm wide polyester mesh strips (Scrynel PET230 HC), 50 microns thick, applied over the top of the paper sensors. These strips were packaged with absorbent packs of silica gel.

### Example 2

1. Glucose test strips in accordance with the invention were made in accordance with Example #1

2. Blood is dropped on plasma separator which prevents red blood cell leakage to the reaction area. A color signal generated by the said enzymatic reaction in the presence of glucose and is read by the glucometer provided and converted into glucose value in mg/dL or mmol/L.

3. The final color on the membrane is read using a specifically designed Glucometer which houses an LED, an optical sensor, a microprocessor that receives digital signals from the optical sensor and converts the frequency values to glucose concentration based on preset algorithm incorporated in the microprocessor.

4. The glucometer allows for storing 50 previous values and also displays errors in case the strip is inserted in a wrong manner or does not have enough blood on the strip for reliable reporting of blood glucose values

5. As opposed to all other glucometers the detection of glucose is not in whole blood, but is in plasma

SOP—Standard Operating Procedure

1. Wash hands with warm water & soap and dry them well before you start testing.

2. Take a strip from the container. Close the container again immediately

3. Insert a new lancet into the lancet holder. Lancet must audibly click into place

4. Press the priming button as far as it will go. The device is primed when a yellow dot appears in the middle of the release button

5. Prick finger with the lancet provided after wiping with an alcohol swab

6. Apply the drop of blood to the centre of the orange field on the strip & put aside

7. Turn on the machine; wait for the prompt ‘INSERT STRIP’

8. Wipe off excess blood, if any, with tissue. On seeing the prompt, insert strip in the slot provided with blood drop side being the lower side.

9. Wait for the glucose reading to appear on the screen. The readings get saved automatically

10. Turn off the machine after use to conserve battery.

Precautions:

1. Do not use your test strips after the expiry date printed on the container. Discard them in a regular trash can.

2. Dispose off used lancets so that the needles cannot cause inadvertent injury.

Comparison with standard liquid assay for plasma glucose:

The accuracy of the developed test is comparable to other glucose concentration analyzing instruments available in market and is in compliance with ISO 15197 with more than 95% readings with +/−20% of the reference test. N=257; r²=0.88.

1. A test strip for holding reagents to determine blood glucose level, said strip comprising
a) a plasma separator membrane which is a composite membrane for separation of plasma from whole blood, said plasma separator membrane comprising an enhancer mix of metallic ions, which enhances a color developed due to an enzymatic reaction of an analyte with an enzyme mix;
b) a reaction membrane comprising the enzyme mix specific to the analyte, said analyte being glucose; and
a chromogenic substrate which reacts specifically with a product of the enzymatic reaction of the analyte with the enzyme mix.

2. The test strip as claimed in claimed in claim 1, wherein said plasma separator membrane is made up of polysulphone, cellulose, nitrocellulose and glass fibres, preferably polysulphone.

3. The test strip as claimed in claim 1, wherein said composite membrane has specific dimensions and is positioned in said test strip for the purpose of plasma separation from the whole blood.

4. The test strip as claimed in claim 1, wherein said enzyme mix comprises a glucose oxidase and a peroxidase.

5. The test strip as claimed in claim 1, wherein said chromogenic substrate is specific to the product of said enzymatic reaction and develops a colored product by reacting with the product of said enzymatic reaction.

6. The test strip as claimed in claim 5, wherein said chromogenic substrate is selected from the group of compounds 3,3′,5,5′-Tetramethylbenzidine, 2,2′-azino-bis (3-ethylbenzothiazoline-6-sulphonic acid),3,3′-diaminobenzidine and o-Phenylenediamine.

7. The test strip as claimed in claim 5, wherein said enhancer mix enhances the of said colored product.

8. The test strip as claimed in claim 1, wherein the enhancer mix is present in dried form in the plasma separator membrane.

9. The test strip as claimed in claim 1, wherein said enhancer mix is a metal salt selected from a group consisting of copper sulfate, nickel chloride, nickel ammonium sulfate and cobalt chloride.

10. A process for the preparation of the test strip, comprising the steps of
preparing a mix of glucose oxidase and peroxidase in a citrate buffer, spreading the mix on a cellulose sheet,
drying the cellulose sheet, adding a substrate on the mix,
drying the substrate to obtain a reaction strip,
preparing a solution of an enhancer in a buffer, and
quenching a membrane with the enhancer.
drying the membrane to obtain a plasma separator membrane, placing said reaction strip below said plasma separator membrane to obtain a sandwich, and by placing said sandwich on an additional membrane to obtain the test strip.

11. The process as claimed in claim 10, wherein said citrate buffer has a pH between 5 to 6, such as citrate buffer.

12. The process as claimed in claim 10, wherein said additional membrane is comprises a porous fabric or material such as polyamide, polyolefin, polysulfone, or cellulose.
